(19) United States
(12) Patent Application Publication
(10) Pub. No.: US 2026/0354917 A1
(43) Pub. Date: Nov. 12, 2026

(54) SURGICAL ROBOTIC SYSTEM WITH SINGLE PORT ACCESS SYSTEM

(71) Applicant: COVIDIEN LP, Mansfield, MA (US)

(72) Inventors: Alexander D. Norman, Cambridge (GB); Michael J. Blom, Cambridge (GB); Andrew G. Forbes, Cambridge (GB); Charles F. Kilby, Cambridgeshire (GB); Gary Stacey, Cambridge (GB)

(21) Appl. No.: 19/131,616

(22) PCT Filed: Nov. 28, 2023
(86) PCT No.: PCT/US2023/081251

§ 371 Date: May 21, 2025

Related U.S. Application Data
(60) Provisional application No. 63/428,567, filed on Nov. 29, 2022.

(51) Int. Cl. A61B 34/37; A61B 90/00; A61B 90/50

(52) U.S. Cl. CPC A61B 34/37; A61B 90/37; A61B 90/50; A61B 2090/5025; A61B 2090/506; A61B 2090/508

(57) ABSTRACT

A surgical robotic system includes a control drive unit having a plurality of instrument drive units and a surgical instrument and a camera drive unit. The system also includes a plurality of instruments each of which is coupled to one instrument drive unit and a camera coupled to the camera drive unit. The system further includes a surgical port assembly configured to receive the plurality of instruments and the camera and a setup arm having a plurality of links. The setup arm is coupled to the control drive unit and has a constrained remote center of motion.

### CROSS-REFERENCE TO RELATED APPLICATIONS

This application claims the benefit of, and priority to, U.S. Provisional Patent Application Ser. No. 63/428,567 filed on Nov. 29, 2022. The entire contents of the foregoing application is incorporated by reference herein.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body.

### SUMMARY

According to one embodiment of the present disclosure, a surgical robotic system is disclosed. The surgical robotic system includes a control drive unit having a plurality of instrument drive units and a camera drive unit. The system also includes a plurality of instruments each of which is coupled to one instrument drive unit and a camera coupled to the camera drive unit. The system further includes a surgical port assembly configured to receive the plurality of instruments and the camera, and a setup arm having a plurality of links. The setup arm is coupled to the control drive unit and has a constrained remote center of motion.

Implementations of the above embodiment may include one or more of the following features. According to one aspect of the above embodiment, the setup arm may further include a parallelogram joint configured to mechanically constrain the remote center of motion. The setup arm may also include a rotatable joint coupled to the parallelogram joint. The rotatable joint may be disposed along a vertical axis passing through the remote center of motion. The parallelogram joint may be configured to constrain pitch motion of the control drive unit from about +10 degrees to about −50 degrees. The surgical port assembly may be disposed at least partially at the remote center of motion. The setup arm may include an L-shaped link having a first joint coupled to the setup arm and a second joint coupled to the control drive unit. The first joint may be configured to control yaw motion of the control drive unit and the second joint may be configured to control pitch motion of the control drive unit. Each of the first joint and the second may include an actuator. The controller may be configured to control the actuators to constrain the pitch motion and the yaw motion of the control drive unit.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a movable cart according to an embodiment of the present disclosure;

FIG. 2 is a perspective view of a surgical robotic arm of the surgical robotic system of FIG. 1 according to an embodiment of the present disclosure;

FIG. 3 is a perspective view of a movable cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5A is a perspective view of a movable cart having a setup arm with a multiple instrument drive units according to one embodiment of the present disclosure;

FIG. 5B is a perspective view of the movable cart of FIG. 5B with covers removed from the setup arm according to one embodiment of the present disclosure;

FIG. 6 is a perspective view of a movable cart having a setup arm with a multiple instrument drive units according to another embodiment of the present disclosure; and

FIG. 7 is a perspective view of a control drive assembly for controlling a plurality of surgical robotic instruments and a camera according to another embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more movable carts having a surgical robotic arm coupled to a setup arm. The surgeon console receives user input through one or more interface devices, which are processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement command and to generate a torque command for activating one or more actuators of the robotic arm, which would, in turn, move the robotic arm in response to the movement command.

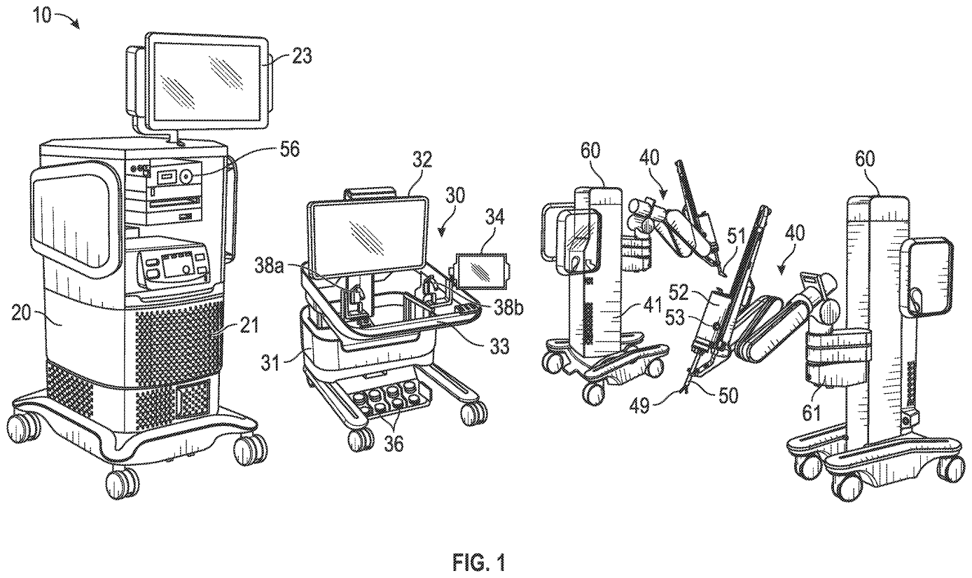

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 removably coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first display 32, which displays a video feed of the surgical site provided by camera 51 of the surgical instrument 50 disposed on the robotic arm 40, and a second display 34, which displays a user interface for controlling the surgical robotic system 10. The first display 32 and second display 34 may be touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

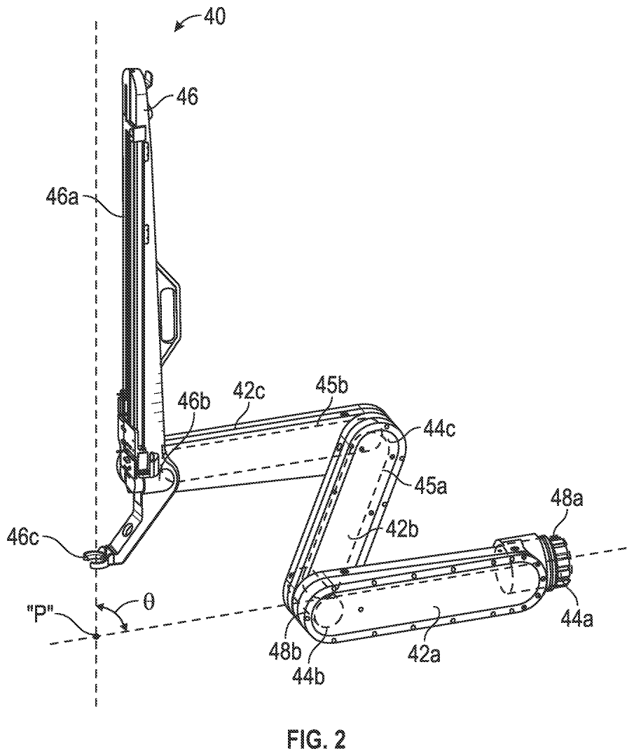

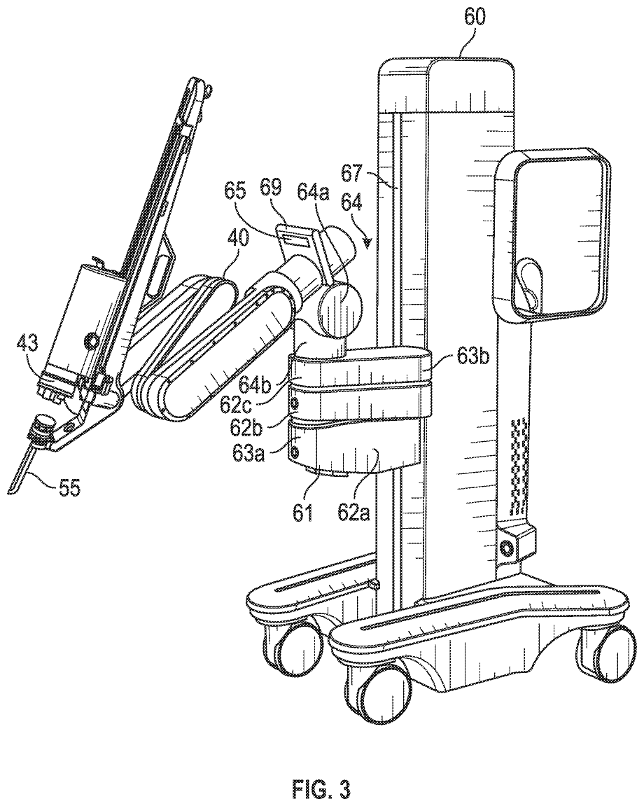

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61. The movable cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

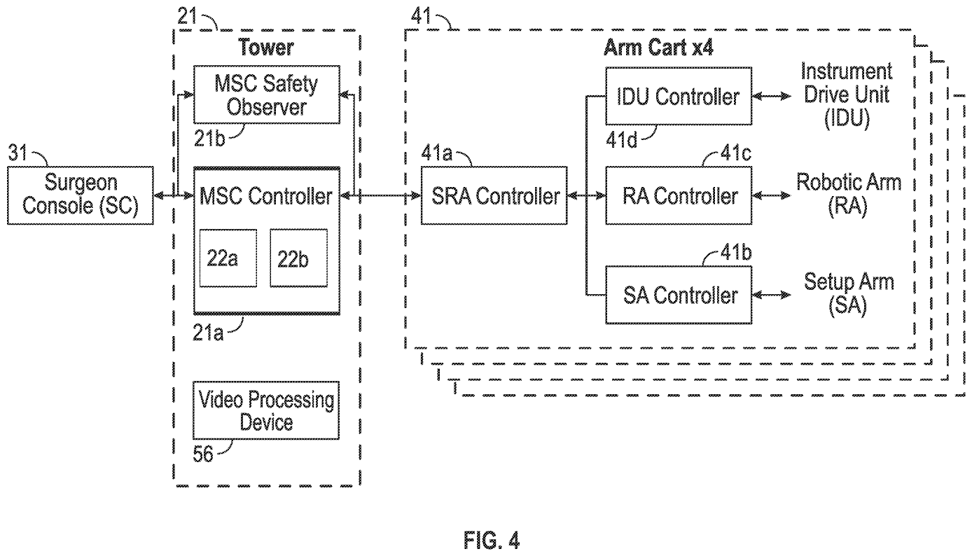

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

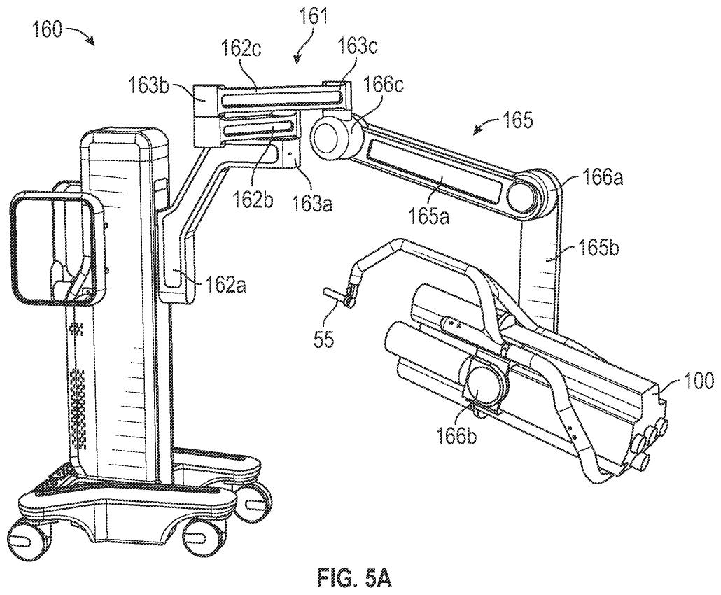

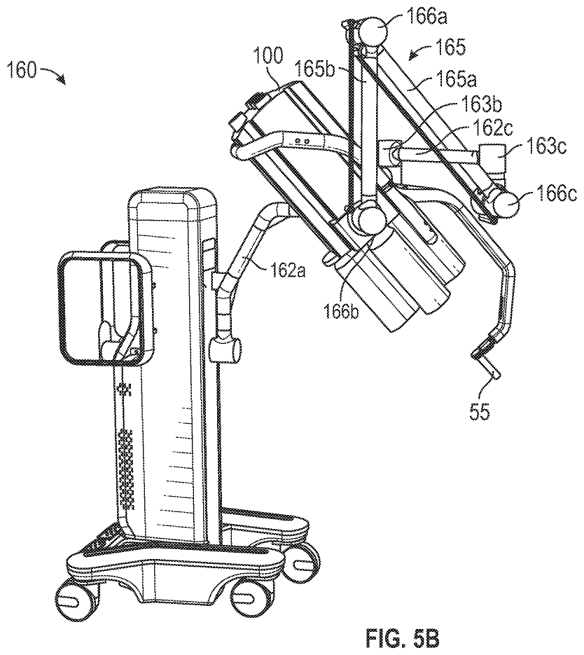

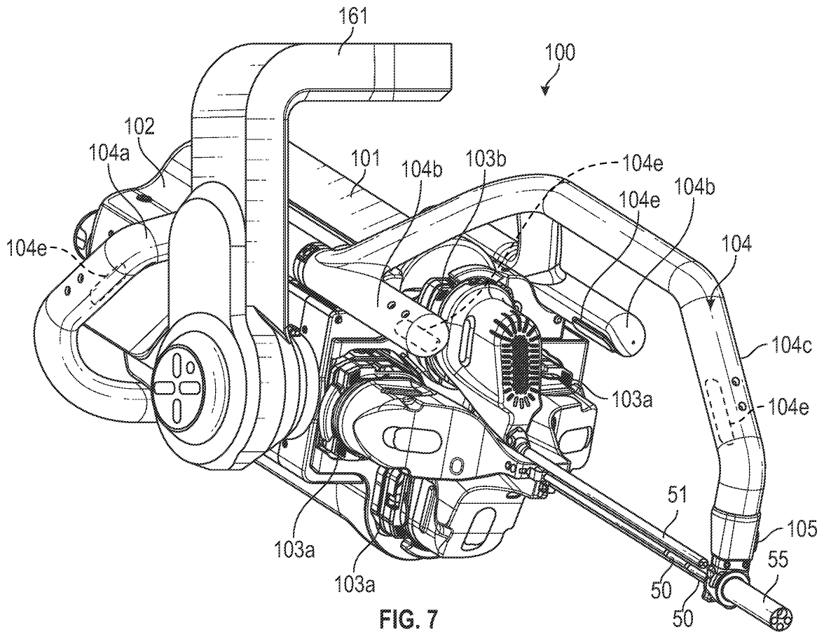

With reference to FIG. 5A, the robotic system 10 may also be used with a multiple instrument movable cart 160 including a control drive assembly 100 configured to actuate a plurality of instruments 50 and the camera 51. Thus, as compared with the robotic arms 40 each of which controls a single instrument 50 that is inserted through a corresponding access port 55, the movable cart 160 is used with a surgical port assembly 16 (FIG. 7) which can serve as a single access port for multiple instruments 50 and the camera 51.

With reference to FIG. 7, the control drive assembly 100 is pivotably mounted to a setup arm 161 of the movable cart 160. Control drive unit 101 of control drive assembly 100 includes a housing 102 that supports instrument drive assembly 103, including instrument drive units 103a (e.g., three) and camera drive unit 103b. The instrument drive units 103a are coupled to instruments 50 and the camera drive unit 103b is coupled to the endoscopic camera 51. The instruments 50 and the camera 51 are inserted into the surgical port assembly 16 having a plurality of lumens as shown in FIG. 7 of any suitable shape or size configured for passage of the instruments 50 and the camera 51 through individual corresponding lumens. While four lumens are illustrated, it should be understood that more or less lumens may be provided. The surgical port assembly 16 is configured to be inserted into the patient.

Control drive unit 101 further includes a support bar assembly 104 that is mounted to housing 102. Support bar assembly 104 may have a hollow construction configured to enable internal wiring and weight reduction. Support bar assembly 104 includes a rear bar 104a having a U-shaped configuration that extends around sidewalls of a proximal portion of housing 102 and beneath housing 102. The support bar assembly 104 also includes handles 104b, and a port arm 104c that extends from handles 104b distally to port latch assembly 105 on a distal end portion of port arm 104c. Port arm 104c has an arched configuration and extends to a float on a proximal end portion of port latch assembly 105 to enable post manufacturing alignment of port latch assembly 105 and to help mitigate any assembly stack up errors. Port latch assembly 105 is configured to secure the surgical portal assembly 16 and defines the RCM. Support bar assembly 104 further includes a plurality of brake release buttons 104e on inner facing surfaces of rear bar 104a, handles 104b, and port arm 104c of support bar assembly 104 that are configured to stop linear movement of instrument drive and/or camera drive units 103a, 103b relative to control drive unit 101. The control drive assembly 100 and its operation are described in further detail in U.S. Provisional Patent Application No. 63/341,459 filed on May 13, 2022, the entire contents of which are incorporated by reference herein.

With reference to FIG. 5A, the movable cart 160 includes a setup arm 161 having a first link 162a that is vertically movable allowing for height adjustment of the setup arm 161. The setup arm 161 also includes second and third links 162b and 162c. The second link 162b is coupled to the first link 162a via a rotational joint 163a and to the third link 162c via a second rotational joint 163b. The joints 163a and 163b may be passive allowing for manual adjustment of the setup arm 161. The setup arm 161 also includes a combined parallelogram joint 165 having a pair of links 165a and 165b rotationally coupled via a joint 166a. The control drive assembly 100 is coupled to the link 165b via a joint 166b. The parallelogram joint 165 is rotationally coupled to a third joint 163c of the setup arm 161 via a joint 166c. The joint 163c of the setup arm 161 and the joints 166a-c may be active and/or spring counterbalanced. As used herein, the term “active joint” denotes a joint having an actuator, e.g., motor. The configuration of the parallelogram joint 165 provides for a mechanically constrained RCM.

The third joint 163c of the setup arm 161 and the joint 166b of the parallelogram joint 165 are placed directly over the RCM. Thus, the joint 163c alone sets the yaw of the control drive assembly 100. To ensure that the third joint 163c remains over (i.e., along a vertical axis perpendicular to the floor) the RCM, the parallelogram joint 165 provides pitch motion of the control drive assembly 100, which may be from about +10° to about −50° with a mechanically constrained RCM. The other joints 163a and 163b may remain passive, as they are not engaged during repositioning. The vertical stage, i.e., the first link 162a, can also remain passive, as it is only used for manual setup. In embodiments, the first link 162a may be spring counterbalanced to address issues of high inertia.

With reference to FIG. 5B, the links 162a-c of the setup arm 161 and the links 165a-b of the parallelogram joint 165 may be rigid links rather than belt-driven. The rigid links may be carbon fiber reinforced plastic tubes, which are very rigid in bending and torsion. Cast and machined metal nodes may be used at the joints 163a-c and 166a-c. While a linkage-driven parallelogram is more limited in its rotation angles, it is significantly stiffer, simpler to assemble, and leaves more internal space for wiring These features make this design well suited to the single port robotic system which has long and highly loaded links but only has approximately 60 degrees of travel.

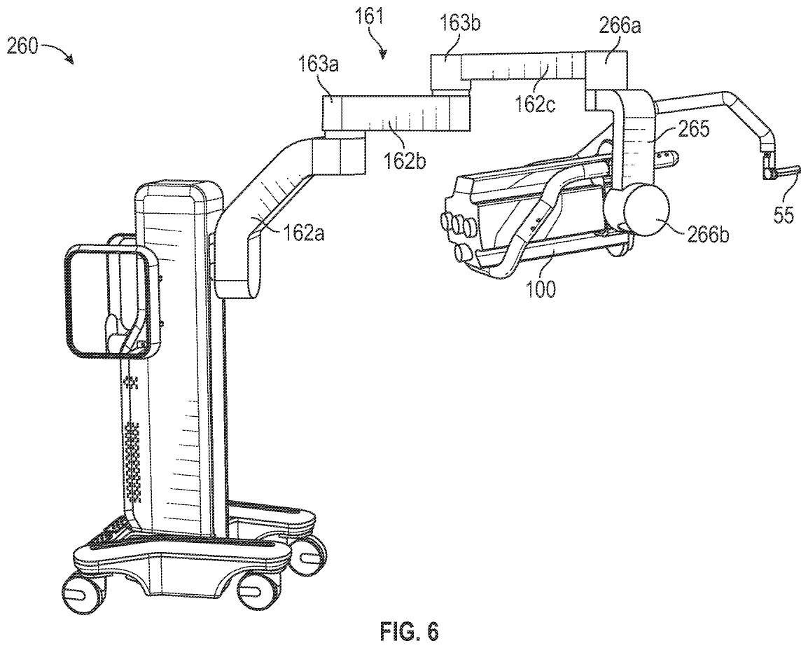

With reference to FIG. 6, another embodiment of a multiple instrument movable cart 260 which is substantially similar to the movable cart 160 with the exception of the parallelogram joint 165, which is replaced by a L-shaped link 265. The setup arm 161 is coupled to the L-shaped link 265 via a first joint 266a providing rotation about a first axis, e.g., Y axis. The L-shaped link 265 is coupled to the control drive assembly 100 via a second joint 266b providing rotation about a second axis, e.g., X axis, that is perpendicular to the first axis. The first joint 266a and the second joint 266b are controlled by the controller 21a to limit pitch and yaw motion thereby controlling RCM, i.e., software constrained RCM.

In order to maintain an RCM while making pitch adjustments, the first link 162a, the first joint 163a, and the second joint 163b are also motorized and controlled by the controller 21a. To minimize inertia and enable an easily back-drivable system for manual setup, the joints of the setup arm 161 may be spring-counterbalanced. In further embodiments, the actuators may be back-drivable.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a control drive unit including a plurality of instrument drive units and a camera drive unit;
a plurality of instruments each of which is coupled to one instrument drive unit;
a camera coupled to the camera drive unit;
a surgical port assembly configured to receive the plurality of instruments and the camera;
a setup arm including a plurality of links, the setup arm coupled to the control drive unit and having a constrained remote center of motion.

2. The surgical robotic system according to claim 1, wherein the setup arm further includes a parallelogram joint configured to mechanically constrain the constrained remote center of motion.

3. The surgical robotic system according to claim 2, wherein the setup arm includes a rotatable joint coupled to the parallelogram joint.

4. The surgical robotic system according to claim 3, wherein the rotatable joint is disposed along a vertical axis passing through the constrained remote center of motion.

5. The surgical robotic system according to claim 4, wherein the parallelogram joint is configured to constrain pitch motion of the control drive unit from about +10° to about −50°.

6. The surgical robotic system according to claim 1, wherein the surgical port assembly is disposed at least partially at the constrained remote center of motion.

7. The surgical robotic system according to claim 1, wherein the setup arm includes an L-shaped link that includes a first joint coupled to the setup arm and a second joint coupled to the control drive unit, the first joint configured to control yaw motion of the control drive unit and the second joint configured to control pitch motion of the control drive unit.

8. The surgical robotic system according to claim 7, wherein each of the first joint and the second joint includes an actuator.

9. The surgical robotic system according to claim 8, further comprising a controller coupled to the actuators, wherein the controller is configured to control the actuators to constrain the pitch motion and the yaw motion of the control drive unit.
